(19) United States
(12) Patent Application Publication
(10) Pub. No.: US 2007/0010710 A1
(43) Pub. Date: Jan. 11, 2007

(54) Biological imaging systems

(76) Inventor: Timothy Perez, Albuquerque, NM (US)

Correspondence Address: V. Gerald Grafe, esq., P.O. Box 2689, Corrales, NM, 87048, US

(21) Appl. No.: 11/482,359

(22) Filed: Jul. 7, 2006

Related U.S. Application Data
(60) Provisional application No. 60/698,207, filed on Jul. 9, 2005.

(51) Int. Cl. A61B 1/04

(52) U.S. Cl. 600/118; 600/109

(57) ABSTRACT

The present invention includes a laparoscopic imaging apparatus including a housing mountable in a known relationship to a patient, an image capture element mounted with the housing, and an image control system connected to the image capture element. The image capture element is mounted with the housing such that when in use, the image capture element is within the body of the patient, and further such that the image capture element is adapted to generate a signal representative of an image of portions of the volume within the body of the patient. The image control system is adapted to be responsive to the signals generated by the image capture element as well as control inputs provided by a user. The image control system is further adapted to select a portion of an image represented by the signal, wherein the portion of the image is less than the whole image.

### CROSS-REFERENCE TO RELATED APPLICATIONS

The present application claims priority under 35 U.S.C. 119 to provisional application Ser. No. 60/698,207, filed Jul. 9, 2005.

### FIELD OF THE INVENTION

This invention relates generally to the field of surgery, medical devices and optical instruments, and in particular to systems and devices for minimally invasive surgery and observation within the abdominal cavity.

### BACKGROUND OF THE INVENTION

Laparoscopy (visualization of the abdominal cavity) has been performed since the beginning of the 20th century. FIG. 1 shows the relative configuration of a present laparoscopic system 1 and an abdominal cavity 100, including an object of interest 120 therein. A rigid lens 4 is introduced through the body wall with the support of a trocar 2, which allows a surgeon to look inside the abdominal cavity 100. This technique was limited initially since the surgeon had to put his eye to lens and was mainly for diagnostic purposes and very simple procedures. The revolution in laparoscopy came in the 1980's with the introduction of a video camera 6, to which light from the rigid lens 4 could be transmitted through a body portion 3. A video camera 6 could be coupled to the body portion 3 and a monitor 7 having a display 8. Light passing through the rigid lens 4 is captured by the video camera 6 and transmitted to the monitor 7 in the form of an image. With the entire surgical team able to see the image, this opened the way for performing increasingly complex operations.

Despite almost a century of use, there remain numerous significant problems with the instrumentation available for laparoscopic surgery and observation. During an open procedure the surgeon visualizes the target area directly through an incision in the body wall. The variables of image quality are surgeon eyesight, lighting and retraction of surrounding structures. In laparoscopic surgery the image quality is influenced by many more factors. In essence, the surgeon is operating on a video image rather than on the body. Issues that influence that image include (1) Surgeon position and orientation; (2) Camera manipulation; (3) lens properties; and (4) the camera, monitor, and cabling system.

During open surgery the target organ and surgeon's head comprise the 2 points of a straight line. Present technology still considers laparoscopy to be a “line of sight” practice, similar to open surgery. However, with laparoscopic surgery there are 3 or 4 points between the target and surgeon. Ideally the operative field, laparoscope, monitor, and surgeon should be in the same vertical plane. This is not always possible and the deviation from this alignment distorts the surgeon's view. In order to compensate for this distortion, the surgeon must mentally accommodate for the different variables. Conventional technology provides digital images, but does not provide any compensation for the various forms of visual distortion.

The camera holder is the person who holds the laparoscope during the surgery. Typically, it is an assistant surgeon or a surgical scrub tech. The camera operator must visualize the precise part of the body with the correct orientation and perspective in order to substitute for line of sight visualization. As shown in FIG. 2, the laparoscope may be adjusted to track the surgeon's movements. For example, the rigid lens 4 may be reoriented by translating a body portion 3 along the arrow A to accommodate a surgeon's request. However, as a cylindrical laparoscope has no fixed rotational orientation, there can be a tendency for the instrument to roll or rotate about its longitudinal axis defined by the arrow B, thus potentially skewing the orientation of the image displayed on the monitor 7. Thus any movement of the laparoscope is potentially problematic, as there are a number of degrees of freedom in the conventional system, and it is a taxing effort to maintain the proper horizontal and rotational orientation of the laparoscope and the resulting image used by the surgeon. The mental and physical fatigue associated with operating the laparoscope can degrade accuracy of the camera holder thus slowing the operation or procedure.

A conventional laparoscope is a rigid, tubular optical lens inserted through a trocar into the abdominal cavity. Laparoscopes vary from 3 mm to 12 mm in diameter, and have a limited field of view 9 as shown in FIG. 2. The end of the lens can be angled from 0° to 75°, which enables “looking around corners”. But while advances in light transmission technology, etc. have occurred, the basic laparoscope has changed little in the last 50 years. A conventional laparoscope's utility is wholly dependent on how well it is used. This leads to several significant weaknesses noted above, including: (1) a conventional laparoscope is a round tube inserted through a round opening, creating a tendency to deviate from the horizontal thus skewing the image; (2) the imaging surface of the lens is small which limits the amount of pixel data gathered; (3) the small lens area is prone to soiling from water vapor, blood, etc, and needs to be cleaned frequently.

Prior advances in laparoscope technology have been limited to imaging technology. As video technology improves the lines of resolution have increased, providing an image with better detail and color. Presently all laparoscope images are intended for 3×4 format monitors, like a contemporary television. However, laparoscopic surgery is not “watching television”. The image being “operated on” should be approximate what a surgeon would see if they could be looking from within the abdominal cavity. Instead of enhancing the surgeon's visual information, conventional laparoscopes inadvertently often contribute to its limitation. Accordingly, there is a need for new laparoscopic technology that addresses the problems with current technology.

### SUAMMRY OF THE INVENTION

The present invention provides several methods and apparatuses that correct deficiencies in current laparoscopes, and that provide new approaches to enhancing the information presented to the surgeon in laparoscopic surgery. The present invention includes a laparoscopic imaging apparatus including a housing mountable in a known relationship to a patient, an image capture element mounted with the housing, and an image control system connected to the image capture element.

The housing is adapted to be at least partially inserted within the body of the patient, and can function as its own insertion device thus eliminating the need for a separate trocar. The image capture element is mounted with the housing such that when in use, the image capture element is within the body of the patient, and further such that the image capture element is adapted to generate a signal representative of an image of portions of the volume within the body of the patient. The image control system is adapted to be responsive to the signals generated by the image capture element as well as control inputs provided by a user. The image control system is further adapted to select a portion of an image represented by the signal, wherein the portion of the image is less than the whole image.

In various example embodiments described in more detail below, the image control system may be adapted zoom-in, zoom-out and pan in various directions through the image in response to user inputs. The image control system can also permit a user to view to distinct portions of the image, such as for example two views of the same volume, including for example views of the same volume from different angles or with different zooming effects. All functional aspects of the image control system are performed on the image represented by the signals from the image capture element, and are accomplished without any need for the user to manipulate or maneuver the imaging apparatus relative to the patient's body. Other alternative features of the present invention, such as a display system and voice input system, are described in greater detail below with reference to the Figures.

### BRIEF DESCRIPTION OF THE FIGURES

FIG. 1 is a schematic cross-sectional illustration of the relative configuration of a prior art laparoscopic system and a patient's abdominal wall.

FIG. 2 is a schematic cross-sectional illustration of the operational limitations of the prior art laparoscopic system.

FIG. 3 is a schematic cross-sectional illustration of an imaging system according to the present invention.

FIG. 4 is a schematic representation of elements of the imaging system of the present invention.

FIG. 5 is a schematic representation of elements of the imaging system of the present invention.

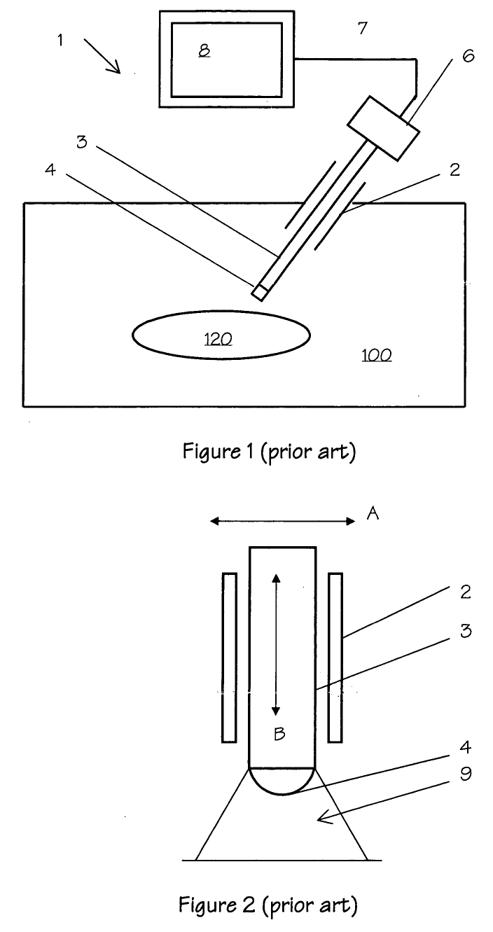

### DETAILED DESCRIPTION OF THE PREFERRED EMBODIMENTS

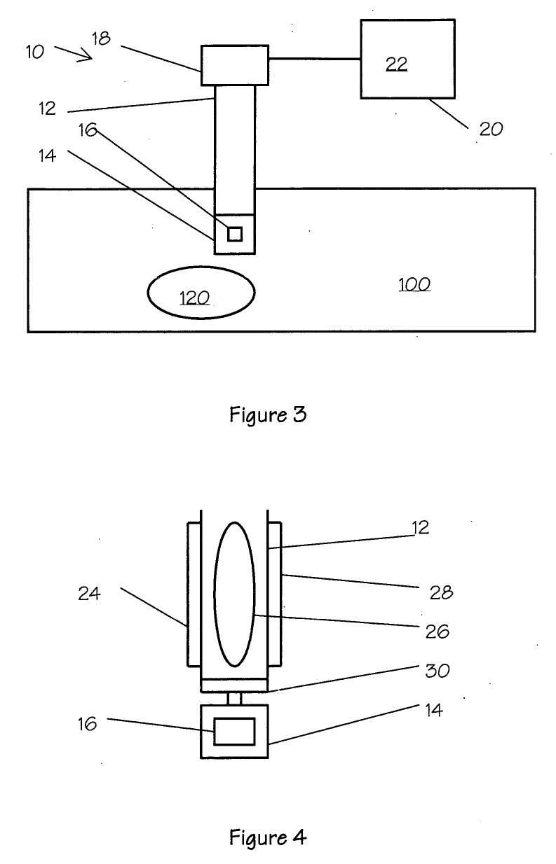

As shown in FIGS. 3 through 5, the present invention includes a laparoscopic imaging apparatus 10 adapted to provide a number of improvements over the prior art. An example imaging apparatus 10 includes a housing 12, mountable in a known relationship to a patient, an image capture element 14 mounted with the housing 12, and an image control system 18 connected to the image capture element 14.

The example housing 12 is adapted to be at least partially inserted within the body of the patient, and can function as its own insertion device thus eliminating the need for a separate trocar. In other variations from the example embodiment, the housing 12 is adapted to be substantially fixed relative to the skin of the patient, thus minimizing any movement of the imaging apparatus 10 and reducing the risk of any damage to the tissues surrounding the incision through which the imaging apparatus is 12 inserted into the patient's body. As used herein, the term skin refers to the epidermis and any underlying adipose tissue, fascia, or other connective tissue normally found within the body of a patient.

The example image capture element 14 is mounted with the housing 12 such that when in use, the image capture element 14 is within the body of the patient, and further such that the image capture element 14 is adapted to generate a signal representative of an image of portions of the volume within the body of the patient. The example image control system 18 is adapted to be responsive to the signals generated by the image capture element 14 as well as control inputs provided by a user. The example image control system 18 is further adapted to select a portion of an image represented by the signal, wherein the portion of the image is less than the whole image.

The example image capture element 14 can be any device that functions to receive electromagnetic radiation and convert electromagnetic radiation into an electrical, optical or electro-optical signal representative of the image of portions of the volume within the body of the patient. An example of a device suitable as an image capture element 14 includes a complementary-metal-oxide semiconductor (CMOS) photodetector, which is known in the optical arts. Other suitable devices include one or more charge coupled devices (CCD) and all manner of photodetectors, photodiodes and the like that are adapted to receive electromagnetic radiation across a portion of the spectrum (including, as examples, infrared radiation, radiation visible to the human eye, and ultraviolet radiation). The image capture element 14 can include an array 16 of one or more of the devices suitable for receiving electromagnetic radiation, and the array 16 can be arranged in two- or three-dimensions for receiving electromagnetic radiation across a substantially large solid angle within the body of the patient. Alternatively, liquid lens technology can be used to accommodate a wide field of view, controllable zooming, controllable panning, and controllable angled viewing without loss of image quality. See, e.g., http://www.varioptic.com/en/; Wired magazine, Issue 13.04, April 2005.

The example image control system 18 includes the necessary hardware and software that are functionally responsive to the signals generated by the image capture element 14 as well as control inputs provided by a user. The image control system 18 can include, for example, a controller or processor, signal conditioning circuitry and memory capacity for receiving, storing and outputting various portions of the image represented by the signal received from the image capture element 14. The image control system 18 also can be adapted to perform a number of other functions to aid an operator in the performance of a surgery or procedure.

For example, in one example embodiment, the image control system 18 can be adapted to select a sequence of portions of the image, where the portion selected at the beginning of the sequence provides an initial portion, and wherein each other portion in the sequence comprises an offset along a line through the image from the previous image. This functionality provides a user with a panning effect provided by the image control system 18, thus permitting the user to effectively change the field of view across a plane substantially perpendicular to the housing 12 without having to change the position of the housing 12 relative to the patient's body.

In another example embodiment, the image control system 18 is adapted to select a sequence of portions of the image, where the portion selected at the beginning of the sequence provides an initial portion, and wherein each other portion in the sequence comprises a portion of the previous portion in the sequence. This functionality provides a user with a zoom-in effect provided by the image control system 18, thus permitting the user to effectively change the field of view along an axis substantially parallel to the housing 12 without having to change the position of the housing 12 relative to the patient's body. Similarly, the image control system 18 can be adapted to select a sequence of portions of the image, where the portion selected at the beginning of the sequence provides an initial portion, and wherein each other portion in the sequence comprises a portion of the image that is a superset of the previous portion in the sequence. This functionality provides a user with a zoom-out effect provided by the image control system 18, thus permitting the user to effectively change the field of view along an axis substantially parallel to the housing 12 without having to change the position of the housing 12 relative to the patient's body.

The image control system 18 can be further adapted to select more than one portion of the image represented by the signal provided by the image capture element 14. For example, the image control system 18 can be adapted to select first and second portions of the image, one of which includes a subset of the image and the other of which includes a subset of the image or the entire image. In one embodiment, the image control system 18 permits a user to view to distinct portions of the image, such as for example two views of the same volume. Alternatively, the image control system 18 can permit a user to view distinct portions of the image including a zoom-in and a zoom-out image of the same volume, thus providing the user with two views of the same features at distinct relative elevations. As such, the image control system 18 can provide for example a left view and a right view of the same feature, as well as a zoom-in view and a panoramic view of the same feature—without any need for an operator to manipulate or maneuver the imaging apparatus 10 relative to the patient's body.

The image control system 18 can include one or more machine learning algorithms that utilize inductive or deductive reasoning to accurately predict or anticipate a surgeon's request with regards to image processing, storage and output signals. In response to the signals generated by the image capture element 14, the image control system 18 can receive and interpret image data in order to learn the habits and instrument manipulations of the surgeon, the landscape of the patient's tissues and organs, and the appropriate level of resolution of the image during one or more procedures. For example, the image control system 18 may receive data regarding the relative position of the instrument and a portion of the patient's body, and through its machine learning capability, the image control system 18 may automatically zoom in, zoom out, or pan in such a manner that the surgeon's view of the events is enhanced. The learning capacity of the image control system 18 may be aggregated over a number of procedures, or it may be adapted to learn within the context of a single procedure on a single patient. Alternatively, the image control system 18 may incorporate certain learning on an aggregate basis and enhance the historical learning with additional learning developed during each new procedure for each new patient.

In another example embodiment, the imaging apparatus 10 includes a display 20 connected to the image control system 18. The display 20 functions to display a portion of an image 22 selected by the image control system 18. As the image control system 18 can select both first and second portions of the image as described above, the display 20 is adapted to display both the first and second portions of the image selected by the image control system 18. In doing so, the display 20 can include a single element, such as for example a computer monitor, that substantially simultaneously displays both the first and second portions of the image. Alternatively, the display 20 can include a first display element and a second display element, each of which is adapted to display the appropriate portion of the image selected by the image control system 18.

In another variation, the display 20 can include an apparatus worn by the surgeon, such as a pair of goggles, a pair of glasses, a helmet and the like. As before, the display 20 functions to display the first and second portions of the image within the apparatus such that the surgeon feels that he or she is looking directly into the patient without the need for a monitor or other device disposed outside the patient's body.

In another example embodiment, the imaging apparatus 10 includes a locus indicator 24. The locus indicator 24 can be attached to the housing 12, as shown in FIG. 4, for operation within the body of the patient, or it can be disposed on the housing 12 or the image control system 18 for operation outside the body of the patient. The locus indicator 24 functions to identify a region in the volume within the body of the patient that is desired to be imaged such that the image control system 18 selects a portion of the image including the region identified by the locus indicator 24. The locus indicator 24 can include one or more of: a device that tracks a portion of an instrument placed within the body of the patient; a device that tracks the head or eye position of an operator, a device that tracks an optical or electrical signal, such as from a laser, a device that tracks the relationship between two or more instruments, or a device that tracks the position of an organ or tissue within the patient's body.

The locus indicator 24 can be further adapted to be responsive to predetermined signals, motions or signals provided by the surgeon or any instrument trackable by the locus indicator 24. The locus indicator 24 can be adapted to be responsive to a predetermined pivot, shake or repeated motion of a instrument within the patient's body, wherein the predetermined motion of the instrument is indicative of the surgeon's intent to cause the imaging apparatus 10 to operate in a particular fashion. As an example, if the surgeon causes the instrument to move in a predetermined fashion, the locus indicator 24 can be adapted to instruct the image control system 18 to zoom in, zoom out, or pan in a particular direction. Similarly, if the surgeon causes the instrument the move in another predetermined fashion, the locus indicator 24 can be adapted to instruct the control system 18 to perform another function described above.

As noted above, the locus indicator 24 can be disposed outside the body of the patient. For example, the locus indicator 24 may include a radiation detector that is adapted to determine the location of a radiation source associated with an organ or tissue within the patient. In such a manner, a patient may receive a dose of radiation that is adapted to target a tissue or organ subject to the surgery or procedure. The locus indicator 24 is adapted to detect the source of radiation within the patient's body, and further adapted to provide signals to the image control system 18 regarding the location of the radiation such that the image capture element 14 is automatically directed to that tissue or organ. The locus indicator 24 may also include imaging techniques incorporated in stereoscopic surgery that are used to precisely identify the location of a tissue or organ within the patient's body.

Another example imaging apparatus 10 can include an element control system 30 attached to the housing 12 and the image capture element 14. The element control system 30 can be responsive to user input and adapted to move the image capture element 14 relative to the housing 12. The user input can include for example manual or voice commands, or the user input can be automatically determined by the imaging apparatus 10 as a function of the position of a surgical instrument. The element control system 30 can include, for example, both mechanical and electromechanical components that function to move the image capture element 14 relative to the housing 12 in response to user input. For example, the element control system 30 can include a mechanical device, such as a gimbal mounted with the image capture element 14 and the housing 12, as well as associated electronics and signal processing circuitry to operate the gimbal in response to the user input.

The element control system 30 can be adapted to perform a number of functions related to the positioning of the image capture element 14 at the request of a user, including functions such as panning, zooming, assembling and automated positioning of the image capture element 14. For example, the element control system 30 can be adapted to accept a control input to pan, and in response thereto move the image capture element 14 such that the image capture element collects an image offset from the image collected when the control input was received. Alternatively, the element control system 30 can be adapted to accept a control input to scan, and in response thereto move the image capture element 14 to collect a plurality of images that together correspond to substantially all the regions within the patient visible by the image capture element 14. In another example, the element control system 30 can be adapted to store a configuration of the image capture element 14, and accept a control input to return to the stored configuration. In response to the control input to return to the stored configuration, the element control system 30 is adapted to move the image capture element 14 such that the region imaged is substantially the same as the image captured when the image capture element 14 was at the stored configuration.

In another example embodiment, the imaging apparatus 10 includes an illumination system 26 connected to the housing 12. The illumination system 26 functions to direct light into the volume imaged by the image capture element 14, thus providing a portion of the electromagnetic radiation receivable by the image capture element 14. The illumination system 26 can include any light or electromagnetic radiation source, such as for example one or more of light emitting diodes (LED), halogen or xenon bulbs, infrared or ultraviolet emitters, and the like. The illumination system 26 can be adapted to generate electromagnetic radiation in the infrared portion of the spectrum, the ultraviolet portion of the spectrum, or that portion of the spectrum visible to the human eye. The wide spectral range of the illumination system 26 thus permits a user to assess other aspects of the patient's anatomy or physiology that may not be visually apparent. For example, infrared and ultraviolet radiation can be utilized to indicate the viability, oxygenation or analyte presence or concentration within one or more tissues in the patient's body.

In another example embodiment, the imaging apparatus 10 includes an insufflation system 28 connected to the housing 12. The insufflation system 28 functions to pump or divert a gas into a cavity within the body of the patient, thus providing a volume within which the image capture element 14 may function. The insufflation system 28 can include a container or vessel of gas connected to a transmission line, which is routed into the patient's body through or on the housing 12. The insufflation system 28 can use for example, carbon dioxide or another inert gas known in the art of laparoscopy, for creating the cavity within the patient's body. Alternatively, the insufflation system 28 can utilize other gases containing other compounds or elements, such as phosphorous, that are reactive to tissues or fluids within the patient's body for providing a user with information regarding the viability, oxygenation or analyte presence or concentration of those tissues or fluids.

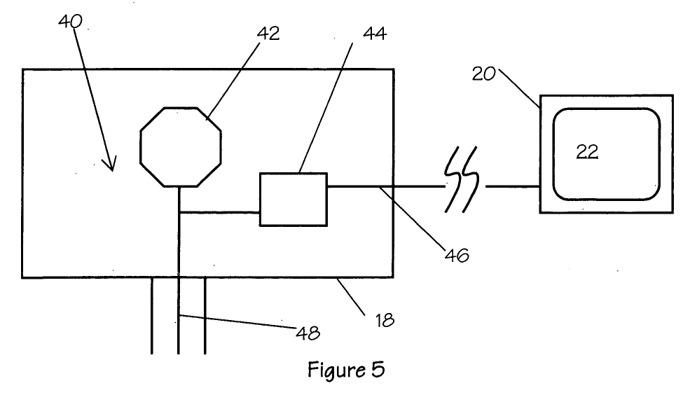

In another example embodiment, the imaging apparatus 10 includes a voice input system 40 connected to the image control system 18. As shown in FIG. 5, the voice input system 40 can include a microphone 42 or other audio input device, a controller 44, a first connector 46 for connecting the controller 44 to the display 20 and a second connector 48 for connecting the controller 44 to additional elements of the present invention. The voice input system 40 functions to generate a command signal corresponding to one of a plurality of voice image control commands such that the image control system 18 selects a potion of the image indicated by the command signal. The plurality of voice image control commands include one or more of a zoom-in command, a zoom-out command, a pan-up command, a pan-down command, a pan-left command, a pan-right command, and a tracking command that commands the image control system 18 to track for example a portion of an instrument within the patient's body.

The voice input system 40 can be connected to other elements of the present invention, such as for example the display 20, the locus indicator 24, the illumination system 26, the insufflation system 28 and the element control system 30. The controller 44 functions to receive voice commands from the user and generate a command signal in response thereto, including for example one or more illumination commands, one or more insufflation commands, one or more tracking commands and one or more control commands. The control commands, for example, can include one or more commands adapted to cause the element control system 30 to move the image capture element 14 relative to the housing 12. Moreover, the controller 44 of the voice input system 40 can be adapted to control the image displayed by the controller in response to one or more display commands, which can include for example commands relating to the orientation or resolution of the images or the portions of the electromagnetic spectrum forming the image.

The present invention further includes a cleaning device (not shown) that functions to clean the image capture element 14 during a procedure. The cleaning device can be attached to the housing 12 and at least partially disposable within the body of the patient. The cleaning device can be operated manually or at the request of the surgeon or operator, or it may be adapted to automatically clean the image capture element 14 in response to a predetermined input. For example, if image data conveyed to the image control system 18 is sufficiently dark or occluded, then the image control system 18 can cause the cleaning device to clean the image capture element 14. A suitable cleaning device includes an apparatus for spraying or depositing water or another suitable fluid on the image capture element 14 in order to remove any excess blood or debris there from. Additionally, the cleaning device can include a pump to remove any excess blood, debris or fluids from the image capture element 14. The pump can be adapted to remove the excess blood, debris or fluids through the projection of air or an inert gas or through the suction or air or an inert gas. The pump can be used in conjunction with, or may be substantially identical to, the insufflation system 28 described above.

The particular sizes and equipment discussed above are cited merely to illustrate particular embodiments of the invention. It is contemplated that the use of the invention may involve components having different sizes and characteristics. It is intended that the scope of the invention be defined by the claims appended hereto.

We claim:

1. A laparoscopic imaging apparatus, comprising:
a housing, mountable in a known relationship to a patient;
an image capture element, mounted with the housing such that when in use the image capture element is within the body of the patient, and adapted to generate a signal representative of an image of portions of the volume within the body of the patient;
an image control system, responsive to said signals, and responsive to control inputs, adapted to select a portion of an image represented by said signal, where said portion is less than all of said image.

2. An apparatus as in claim 1, wherein the image control system is adapted to select a sequence of portions of the image, where the portion selected at the beginning of the sequence provides an initial portion, and wherein each other portion in the sequence comprises an offset along a line through the image from the previous image.

3. An apparatus as in claim 1, wherein the image control system is adapted to select a sequence of portions of the image, where the portion selected at the beginning of the sequence provides an initial portion, and wherein each other portion in the sequence comprises a portion of the previous portion in the sequence.

4. An apparatus as in claim 1, wherein the image control system is adapted to select a sequence of portions of the image, where the portion selected at the beginning of the sequence provides an initial portion, and wherein each other portion in the sequence comprises a portion of the image that is a superset of the previous portion in the sequence.

5. An apparatus as in claim 1, further comprising a locus indicator adapted to identify a region in the volume within the body of the patient that is desired to be imaged, and wherein the image control system selects a portion of the image including the region identified by the locus indicator.

6. An apparatus as in claim 5, wherein the locus indicator comprises one or more of: a device that tracks a portion of an instrument placed within the body of the patient; a device that tracks the head or eye position of an operator; a device that tracks an optical or electrical signal; a device that tracks the relationship between two or more instruments; or a device that tracks the position of an organ or tissue within the patient's body

7. An apparatus as in claim 1, further comprising a voice input system adapted to generate a command signal corresponding to one of a plurality of voice image control commands, and wherein the image control system selects a portion of the image indicated by the command signal.

8. An apparatus as in claim 1, wherein the image control system is adapted to select first and second portions, one of which comprises a subset of the image, and one of which comprises a subset of the image or the entire image.

9. An apparatus as in claim 1, further comprising a display that displays to a user the portion selected by the image control system.

10. An apparatus as in claim 8, further comprising a display system that displays the first and second portions to a user of the system.

11. An apparatus as in claim 10, wherein the display system comprises first and second display elements, wherein the first display element displays the first portion and the second display element displays the second portion.

12. An apparatus as in claim 1, wherein the image capture element collects light having wavelengths in the visible range.

13. An apparatus as in claim 1, wherein the image capture element collects light having wavelengths in the infrared range.

14. An apparatus as in claim 1, wherein the image capture element collects light having wavelengths in the ultraviolet range.

15. An apparatus as in claim 1, wherein the image control system is adapted to store a representation of a portion of an image selected.

16. An apparatus as in claim 1, wherein the image control system is adapted to store a representation of the portion selected, and allows selection of a similar portion of a subsequent image from the image capture element.

17. An apparatus as in claim 1, further comprising an illumination system adapted to direct light into the volume imaged by the image capture element.

18. An apparatus as in claim 1, wherein the illumination system generates light having wavelengths in the visible range.

19. An apparatus as in claim 1, wherein the illumination system generates light having wavelengths in the ultraviolet range.

20. An apparatus as in claim 1, wherein the illumination system generates light having wavelengths in the infrared range.

21. An apparatus as in claim 1, wherein the housing is adapted to mount in a fixed relationship with the skin of the patient.

22. A laparoscopic imaging apparatus, comprising:
a housing, mountable in a known relation to a patient;
an image capture element, mounted with the housing such that image capture element can be moved relative to the housing to collect an image from a plurality of regions within a patient, and generating a signal representative of an image of such a region;
an image control system, responsive to the signal, and adapted to communicate said image or a portion thereof to a user;
an element control system, responsive to user input and adapted to move the image capture element relative to the housing.

23. An apparatus as in claim 22, wherein the image capture element mounts with a gimbal, and the gimbal mounts with the housing.

24. An apparatus as in claim 22, wherein the element control system is adapted to accept a control input to pan, and moves the image capture element such that the image capture element collects an image offset from the image collected when the control input was received.

25. An apparatus as in claim 22, wherein the element control system is adapted to accept a control input to scan, and moves the image capture element to collect a plurality of images that together correspond to substantially all the regions within the patient visible by the image capture element.

26. An apparatus as in claim 22, wherein the element control system is adapted to store a configuration of the image capture element, and is adapted to accept a control input to return to the stored configuration, and moves the image capture element such that the region imaged is substantially the same as the image captured when the image capture element was at the stored configuration.

27. An apparatus as in claim 22, wherein the image capture element is responsive to radiation having wavelengths in the visible range.

28. An apparatus as in claim 22, wherein the image capture element is responsive to radiation having wavelengths in the infrared range.

29. An apparatus as in claim 22, wherein the image capture element is responsive to radiation having wavelengths in the ultraviolet range.

30. An apparatus as in claim 22, further comprising an illumination system adapted to direct light into the volume imaged by the image capture element.

31. An apparatus as in claim 22, wherein the illumination system generates light having wavelengths in the visible range.

32. An apparatus as in claim 22, wherein the illumination system generates light having wavelengths in the ultraviolet range.

33. An apparatus as in claim 22, wherein the illumination system generates light having wavelengths in the infrared range.

34. An apparatus as in claim 22, wherein the housing is adapted to mount in a fixed relationship with the skin of the patient.
